(19) United States
(12) Patent Application Publication
(10) Pub. No.: US 2009/0143418 A1
(43) Pub. Date: Jun. 4, 2009

(54) Compositions and Methods for Preventing and Treating Hair Growth Cycle-Related Conditions

(75) Inventors: Jesse R. Dixon, San Diego, CA (US); Omer H. Yilmaz, Ann Arbor, MI (US); Sean J. Morrison, Ann Arbor, MI (US)

Correspondence Address: Casimir Jones, S.C., 440 Science Drive, Suite 203, Madison, WI, 53711, US

(73) Assignee: THE REGENTS OF THE UNIVERSITY OF MICHIGAN, Ann Arbor, MI (US)

(21) Appl. No.: 12/263,231

(22) Filed: Oct. 31, 2008

Related U.S. Application Data
(60) Provisional application No. 60/991,558, filed on Nov. 30, 2007.

(51) Int. Cl. A61K 31/435; A61K 31/436

(52) U.S. Cl. 514/284; 514/291

(57) ABSTRACT

The present invention relates to compositions and methods for the preventing, treating, and researching discoloration (e.g., hair graying, hair whitening, undesired change in hair color). In particular, the present invention provides compositions and methods for treating, preventing and researching hair discoloration through inhibition of mTOR function (e.g., mTOR activity, mTOR expression). In addition, the present invention provides methods and compositions that utilize mTOR inhibiting agents in the preventing, treating, and researching hair discoloration.

### CROSS-REFERENCE TO RELATED APPLICATIONS

The present application claims priority to pending U.S. Provisional Patent Application Ser. No. 60/991,558, filed 11/30/2007, which is herein incorporated by reference in its entirety.

### FIELD OF THE INVENTION

The present invention relates to compositions and methods for the preventing, treating, and researching hair growth cycle-related conditions (e.g., hair graying, hair whitening, undesired change in hair color, hirsutism, hypertrichosis, undesired hair growth, alopecia, undesired hair shedding, iatrogenically-induced hair changes). In particular, the present invention provides compositions and methods for treating, preventing and researching hair growth cycle-related conditions through inhibition of mTOR function (e.g., mTOR activity, FRAP1/mTOR expression, mTOR protein level, mTOR stability). In addition, the present invention provides methods and compositions that utilize mTOR inhibiting agents in the preventing, treating, and researching hair growth cycle-related conditions.

### BACKGROUND OF THE INVENTION

Hair growth in mammals occurs through a defined process. The follicular life cycle of hair can be divided into 3 phases: anagen, catagen, and telogen. The anagen phase is the phase of active growth. The catagen phase marks follicular regression, and the telogen phase represents a resting period.

Hair growth cycling—that is, the orderly progression through these phases—occurs with characteristic periodicity. In terminal hairs of the human scalp, the anagen phase lasts approximately 3-4 years. The catagen phase lasts approximately 2-3 weeks, and the telogen phase lasts approximately 3 months. On average, approximately 84% of scalp hairs are in the anagen phase, 1-2% are in the catagen phase, and 10-15% are in the telogen phase. There are intrinsic differences in temporal aspects of hair growth cycling between genders (e.g., scalp terminal hair growth cycles last 2-4 years in men and 4-6 years in women; Azziz et al (2000) Endocrin. Rev. 21:347-62; herein incorporated by reference in its entirety), between hair types (e.g., approximately 14-17 months for the short, fine primary vellus hair on the trunk and extremities (Kligman, (1959) J Invest. Dermatol. 33:307-16; herein incorporated by reference in its entirety), and between different sites on the body (e.g., 4 months for eyelashes versus the aforementioned 2-6 years for scalp hairs; Kligman, (1959) J Invest. Dermatol. 33:307-16; herein incorporated by reference in its entirety). There is also a range of hair cycle durations and rates among populations and between ethnic groups.

The temporal characteristics of hair growth cycles can influence the manifestation of hair growth cycling related conditions such as hair graying, hirsutism, hypertrichosis, undesirable hair growth, alopecia, and iatrogenic hair changes. The onset of hair graying, for example, will appear faster in individuals experiencing shorter hair growth cycles than individuals with longer growth cycles, as the replacement of older pigmented hairs with newly-emergent gray hairs will occur more rapidly in the former than the latter. Undesired hair growth occurs in conditions such as hirsutism (in which androgenically-induced patterns of hair growth occur in women such that excessive cutaneous, dark, thick, terminal hair growth is present in locations such as the face and trunk (see, e.g., Elghblawi (2008) Brit. J Nursing 17:192-7; herein incorporated by reference in its entirety). Other disease states or conditions involving undesired hair growth include hypertrichosis (in which there is an overproduction of vellus non-pigmented, non-androgen-responsive hair) or iatrogenic hair changes resulting from pharmaceutical, chemotherapeutic, or radiation treatment. Suppressing or delaying the hair growth cycle can mediate such conditions. Finally, hair shedding or alopecia may also be mediated by manipulation of the hair growth cycle to prevent premature or accelerated loss of existing hairs.

Although there is considerable social, cultural, and psychological significance of hair, at present there are relatively few strategies to address hair growth cycling-related conditions. Some individuals manage the outcome of hair growth cycle-related conditions by utilizing cosmetic or mechanical approaches such as hair dying to address hair graying, hair removal to address undesired hair growth (e.g., shaving, plucking, tweezing, waxing, chemical depilatory agents, laser hair removal, electrolysis), or hair replacement to address hair loss (e.g., wigs, hair transplants). There are significant drawbacks to these strategies, both individually and collectively. Drawbacks include time, expense, ineffectiveness, skin irritation or blistering, and an end result that appears unnatural. Alternatively, some individuals and treating physicians attempt to manage hair growth cycling-related conditions medically. Taking hirsutism as an example, pharmacological treatments are broadly classified as anti-androgen therapy (e.g., flutamide, cyproterone (approved in the US for compassionate use only), dexamethasone, prednisone) or anti-insulin therapy (e.g., metformin). Significant risks and side effects of anti-androgen therapy can include liver toxicity (flutamide), risk of severe infections (dexamethasone, prednisone), and effects intrinsic to androgen level manipulation such as hot flashes, diarrhea, nausea, loss of appetite, loss of libido, and swelling or tenderness of the breasts (gynocomastia). In addition, a recent study by a task force of The Endocrine Society advised against the use of insulin-lowering drugs for the treatment of hirsutism (see, e.g., Martin et al (2008) J Clin Endocrinol Metab. 93(4):1105-20). A drug recently approved for the treatment of hirsutism that inhibits ornithine decarboxylase, Eflornithine, has been reported to be effective in only 30% of patients; side effects include minor skin irritation, folliculitis, stinging, burning, tingling, acne, and/or rash.

Accordingly, there is need in the art for improved methods to modulate the impact of hair growth cycling-related conditions.

### SUMMARY OF THE INVENTION

The present invention relates to compositions and methods for the preventing, treating, and researching hair growth cycling-related conditions (e.g., hair graying, hair whitening, undesired change in hair color, hirsutism, hypertrichosis, undesired hair growth, alopecia, undesired hair shedding, iatrogenically-induced hair changes). The present invention relates to compositions and methods for the preventing, treating, and researching hair growth cycle-related conditions (e.g., hair graying, hair whitening, undesired change in hair color, hirsutism, hypotrichosis, undesired hair growth, prevention of undesired hair shedding). In particular, the present invention provides compositions and methods for treating, preventing and researching hair growth cycle-related conditions through inhibition of mTOR function (e.g., mTOR activity, FRAP1/mTOR expression, mTOR protein level, mTOR stability). In addition, the present invention provides methods and compositions that utilize mTOR inhibiting agents in the preventing, treating, and researching hair growth cycle-related conditions.

Experiments conducted during the course of development of embodiments for the present invention demonstrated effects on hair growth cycle-related conditions (e.g., hair graying, hair growth cycle duration) through inhibition of mTOR function. In particular, inhibition of mTOR function was shown to increase the duration of hair growth cycle resulting in, for example, delayed onset of hair color change (e.g., delayed onset of hair graying) and decreased hair growth. Accordingly, in certain embodiments, the present invention provides methods for treating and/or preventing hair discoloration in a subject, comprising administering to the subject a composition configured to reduce mTOR function (e.g., mTOR activity, FRAP1/mTOR expression, mTOR protein level, mTOR stability) and/or the function of other mTOR pathway members (e.g., IRS1, P13K, PDK1, Akt, PKC, Rac, Rho, LKB1, Rheb, FKBP, mTOR, mLST8/GβL, S6K, S6, 4EBP1, rS6, eIF4E, eIF3, eIF4A, eIF4G, eIF4B, raptor Vps34, rictor, PTEN, GSK3, LKB1, AMPK, RTP801/L, HIF1, REDD1, TSC1, TSC2) within the subject.

The methods are not limited to a particular type of subject. In some embodiments, the subject suffers from hair discoloration. In some embodiments, the subject is at risk for developing hair discoloration (e.g., subject has a genetic predisposition to hair discoloration; subject is over 30 years of age; the subject is at risk for iatrogenically-induced hair graying). The present invention is not limited to particular types of hair discoloration. In some embodiments, hair discoloration includes, but is not limited to, hair graying, hair whitening, and/or a diminishing of a subject's natural hair color.

In some embodiments, the subject suffers from hirsutism. In some embodiments, the subject is at risk for hirsutism (e.g., the subject has a genetic predisposition to hirsutism; the subject has altered androgen levels; the subject has polycystic ovary disease; the subject is obese; the subject suffers from congenital or delayed-onset adrenal hyperplasia; the subject has Cushing syndrome; the subject has hyperinsulinemia; the subject has hyperprolactinemia; the subject has excess growth hormone; the subject has hypothyroidism; the subject is at risk for iatrogenically-induced hirsutism; the subject has one or more ovarian tumors; the subject has one or more Sertoli-Leydig cell tumors; the subject has one or more granulosa-thecal cell tumors; the subject has one or more other tumor that stimulates the ovarian stroma; the subject has an insulin-resistance syndrome; the subject has hyperthecosis; the subject has idiopathic hirsutism).

In some embodiments, the subject suffers from hypertrichosis. In some embodiments, the subject is at risk for hypertrichosis (e.g., the subject has a genetic predisposition to hypertrichosis; the subject is at risk for iatrogenically-induced hypertrichosis due to administration of agents including but not limited to phenytoin, minoxidil, cyclosporine, diazoxide, corticosteroids, phenytoin (Dilantin), streptomycin, hexachlorobenzene, penicillamine, heavy metals, sodium tetradecyl sulfate, acetazolamide, or interferon; the subject is anorexic).

In some embodiments, the subject suffers from accelerated hair shedding. In some embodiments, the subject suffers from alopecia. In some embodiments, the subject is at risk for accelerated hair shedding or alopecia (e.g., the subject is genetically predisposed to hair shedding; the subject is genetically predisposed to alopecia; the subject is at risk for iatrogenically-induced hair shedding; the subject is at risk for iatrogenically-induced alopecia; the subject suffers from androgen imbalance; the subject has nutritional iron deficiency; the subject has papulosquamous diseases of the scalp; the subject is over 30 years of age).

In some embodiments, the subject is experiencing hair growth considered undesirable for cosmetic reasons. In some embodiments, the subject is at risk of hair growth considered undesirable for cosmetic reasons. In some embodiments, the subject is experiencing loss of hair pigmentation considered undesirable for cosmetic reasons. In some embodiments, the subject is at risk of loss of hair pigmentation considered undesirable for cosmetic reasons. In some embodiments, the subject is experiencing hair shedding considered undesirable for cosmetic reasons. In some embodiments, the subject is at risk of hair shedding considered undesirable for cosmetic reasons.

The composition is not limited to a particular manner of reducing mTOR function (e.g., mTOR activity, FRAP1/mTOR expression, mTOR protein level, mTOR stability) within the subject. In some embodiments, the composition reduces mTOR function through inhibition of, activation of, or action on at least one of the following components within the subject: IRS1, PI3K, PDK1, Akt, PKC, Rac, Rho, LKB1, Rheb, FKBP, mTOR, mLST8/GβL, S6K, S6, 4EBP1, rS6, eIF4E, eIF3, eIF4A, eIF4G, eIF4B, raptor, Vps34, rictor, PTEN, GSK3, LKB1, AMPK, RTP801/L, HIF1, REDD1, TSC1, TSC2 (e.g., nucleic acid, mRNA, DNA, protein). The composition is not limited to a particular manner of inhibiting such components. In some embodiments, the composition comprises an mTOR inhibiting agent (e.g., rapamyocin (sirolimus), CCI-779 (temsirolimus), everolimus (RAD-001), AP23573, rapamycin analogs (rapalogs), mTOR antibodies, mTOR siRNAs, agents inhibiting mTOR phosphorylation, agents inhibiting interaction of mTOR with its partners, agents inhibiting interaction of mTOR with its substrates).

The present invention also provides pharmaceutical compositions comprising a pharmaceutically effective amount of an agent that inhibits mTOR function (e.g., mTOR activity, FRAP1/mTOR expression, mTOR protein level, mTOR stability) (e.g., rapamyocin (sirolimus), CCI-779 (temsirolimus), everolimus (RAD-001), AP23573, rapamycin analogs (rapalogs), mTOR antibodies, mTOR siRNAs, agents inhibiting mTOR phosphorylation, agents inhibiting interaction of mTOR with its partners, agents inhibiting interaction of mTOR with its substrates), wherein the pharmaceutically effective amount is sufficient to treat and/or prevent hair growth cycle-related conditions in a subject (e.g., hair graying, hair whitening, undesired change in hair color, hirsutism, hypertrichosis, undesired hair growth, alopecia, undesired hair shedding, iatrogenically-induced hair changes). In some embodiments, the pharmaceutical composition comprises between 1-30 mg/kg of rapamycin (e.g., 1 mg/kg, 2 mg/kg, 3 mg/kg, 5 mg/kg, 10 mg/kg, 15 mg/kg, 20 mg/kg, 29.5 mg/kg rapamycin). In some embodiments, the pharmaceutical composition is configured for topical administration (e.g., transdermal patches, ointments, lotions, creams, gels, drops, suppositories, sprays, liquids, semi-solids, monophasic compositions, multiphasic compositions (e.g., oil-in-water, water-in-oil), foams microspohnges, liposomes, nanoemulsions, aerosol foams, polymers, fullerenes, and powders (see, e.g., Taglietti et al (2008) Skin Ther. Lett. 13:6-8; herein incorporated by reference in its entirety).

In certain embodiments, the present invention provides methods for preventing and/or treating hair growth cycle-related conditions. The present invention is not limited to particular manners of treating and/or preventing hair growth cycle-related conditions. In some embodiments, the methods comprise administering to a subject (e.g., a human being, a dog, a cat, a mouse, an ape, etc.) a composition comprising an agent. The methods are not limited to particular agents. In some embodiments, the agent is designed to increase the duration of a hair's growth cycle. In some embodiments, the agent is an mTOR inhibitor. In some embodiments, the agent is selected from the group consisting of rapamycin and a rapamycin derivative. In some embodiments, the rapamycin derivative is selected from the group consisting of CCI-779, everolimus (RAD-001), and AP23573.

The methods are not limited to particular hair growth cycle-related conditions. In some embodiments, the hair growth cycle-related condition includes, but is not limited to, hair discoloration, hirsutism, alopecia, hypertrichosis, undesired hair growth, and undesired hair loss. In some embodiments, the hair growth cycle-related condition is related to the effects of a medical treatment (e.g., chemotherapy treatment, radiation treatment, and organ transplant treatment). In some embodiments, the hair discoloration is selected from the group consisting of hair graying and hair whitening. In some embodiments wherein the hair growth cycle-related disorder is hirsutism, the hirsutism is related to a condition selected from the group consisting of Polycystic Ovarian Syndrome, Cushing's disease, ovarian tumor, adrenal tumor, congenital adrenal hyperplasia, insulin resistance, excess androgen levels, pregnancy, aging, and obesity.

In some embodiments, the methods further comprise administering at least one additional agent to the subject, wherein the additional agent is selected from the group consisting of an anti-hair discoloration agent (e.g., hair dye), an anti-alopecia agent (e.g., Squaric acid dibutylester and diphencyprone, Cyclosporine (Sandimmune, Neoral), Methoxsalen (8-MOP, Oxsoralen), Anthralin, Clobetasol propionate (Temovate), Prednisone (Deltasone, Meticorten, Sterapred), Triamcinolone acetonide suspension (Kenalog), Betamethasone dipropionate cream 0.05% (Diprosone), Minoxidil (Rogaine), Finasteride (Propecia)), an anti-hirsutism agent (e.g., an oral contraceptive, spironolactone, flutamide, cyproterone, cyproterone acetate, a corticosteroid, finasteride, eflornithine hydrochloride, and metformin), an anti-hair growth agent, an anti-hair loss agent, a hair growth stimulation agent (e.g., Finasteride (Propecia)), and an anti-hypertrichosis agent.

In certain embodiments, the present invention provides compositions comprising an agent designed to increase the duration of a hair's growth cycle and at least one additional agent, wherein the additional agent is, for example, anti-hair discoloration agent (e.g., hair dye), an anti-alopecia agent (e.g., Squaric acid dibutylester and diphencyprone, Cyclosporine (Sandimmune, Neoral), Methoxsalen (8-MOP, Oxsoralen), Anthralin, Clobetasol propionate (Temovate), Prednisone (Deltasone, Meticorten, Sterapred), Triamcinolone acetonide suspension (Kenalog), Betamethasone dipropionate cream 0.05% (Diprosone), Minoxidil (Rogaine), Finasteride (Propecia)), an anti-hirsutism agent (e.g., an oral contraceptive, spironolactone, flutamide, cyproterone, cyproterone acetate, a corticosteroid, finasteride, eflornithine hydrochloride, and metformin), an anti-hair growth agent, an anti-hair loss agent, a hair growth stimulation agent (e.g., Finasteride (Propecia)), and an anti-hypertrichosis agent. In some embodiments, the composition is configured for administration to a subject (e.g., a pharmaceutical composition). For example, in some embodiments, the composition is configured for any type of administration including, but not limited to, through the eyes (ophthalmic), mouth (oral), skin (transdermal, topical), nose (nasal), lungs (inhalant), oral mucosa (buccal), ear, by injection (e.g., intravenously, subcutaneously, intratumorally, intraperitoneally, etc.) and the like. In some embodiments, the agent designed to increase the duration of a hair's growth cycle is an mTOR inhibitor. In some embodiments, the mTOR inhibitor is selected from the group consisting of rapamycin and a rapamycin derivative. In some embodiments, the rapamycin derivative is selected from the group consisting of CCI-779, everolimus (RAD-001), and AP23573.

### BRIEF DESCRIPTION OF THE DRAWINGS

FIG. 1 shows mice irradiated with 11.2 grays followed by bone marrow transplantation as described herein and tracked for 16 weeks. Representative images are shown for each time point.

FIG. 2a shows mice irradiated with 11.2 grays followed by bone marrow transplantation as described herein and treated for 16 weeks with intraperitoneal injections of either rapamycin at 0.4 mg/kg/day or vehicle. Differences in graying were observed. Representative images for each time point are shown. FIG. 2b shows mice irradiated with 11.2 grays followed by bone marrow transplantation as described herein and treated with either rapamycin at 4mg/kg/day or vehicle. Representative images are shown for each time point.

FIG. 3 shows skin sections from DCT-LacZ mice. (A) a DCT-LacZ positive unirradiated control mouse. (B) a DCT-LacZ negative unirradiated negative control mouse. (C) Representative image from a DCT-LacZ positive irradiated mouse. Arrowheads point to follicles that show no X-gal staining. Arrows point to positively stained follicles.

FIG. 4 shows skin sections from DCT-LacZ mice representing different stages of hair growth cycling. In (A) and (B), hair follicles are in the resting telogen phase. In (C) and (D), follicles are in the cycling anagen phase as identified by hairs that are morphologically larger, are extending slightly beyond the epidermis, and have clear black staining indicating ongoing melanin synthesis.

FIG. 5 shows hair regrowth following depilation (in absence of irradiation or bone marrow transplantation) and treatment with either rapamycin (4 mg/kg) or vehicle. Picture sequences from representative mice are shown.

FIG. 6 shows hair regrowth following sublethal irradiation with 650 rads in absence of bone marrow transplantation and treatment with intraperitoneal injections of vehicle (n=5) or 4 mg/kg rapamycin (n=5). Representative pictures from each treatment group are shown at 4, 8, 12, and 16 weeks post-irradiation.

FIG. 7 shows hair regrowth after irradiation with 2×560 rads 3 h apart followed by bone marrow transplantation as described herein. Subsequently, mice were treated with intraperitoneal injections of vehicle or 4 mg/kg rapamycin on alternating days for the first 14 days after transplantation and then daily thereafter. At 4, 8, and 12 weeks post-transplantation, groups of vehicle (n=3 at each time point) and rapamycin-treated mice (n=3 at each time point) were depilated and subsequently not administered treatment. Photos were taken 4 weeks after depilation. Representative pictures from each group are shown.

FIG. 8 shows hair growth following depilation (on day 0) and subsequent application of a 0.5% topical rapamycin ointment (n=5) or vehicle ointment (n=5). Ointment application occurred daily for 7 days.

### DEFINITIONS

To facilitate an understanding of the present invention, a number of terms and phrases are defined below:

As used herein, the term “hair growth cycle-related condition” generally refers to any condition or disease state in which the periodicity or temporal aspect of hair follicle growth cycles (e.g., overall length of time during which the growth cycle progresses from anagen to catagen to telogen phases; duration of each individual phase; existence or duration of a paused, stopped, or intermediate state within or between phases) influences, manifests, causes, manipulates, affects, or impacts the appearance of a hair property. Such hair properties may include but are not limited to presence or absence of hair (without limitation to hair type, be it terminal, vellus, androgenic or non-androgenic), hair pigmentation or reduction or lack thereof, hair thickness, hair diameter, hair density, or hair texture. The overall effect of impacting a hair property may be apparent by considering or assessing a plurality of hairs (e.g., the plurality of terminal hairs of the scalp.) The hairs impacted by hair growth cycle periodicity are without limitation to the anatomical location of hair growth. Examples of hair growth-related conditions include, but are not limited to, hair graying, hair whitening, undesired change in hair color, hirsutism, hypertrichosis, undesired hair growth, alopecia, undesired hair shedding, and iatrogenically-induced hair changes.

As used herein, the term “hirsutism” generally refers to excessive cutaneous, thick, terminal hair growth in places where terminal hair is not normally found.

As used herein, the term “hypertrichosis” generally refers to a non-androgen-related pattern of excessive hair growth that may involve vellus, terminal, or lanugo type hair.

As used herein, “iatrogenically-induced” refers to a condition or disease state caused by a physician, surgeon, or other administering professional or by a medical or surgical treatment (e.g., pharmaceutical treatment, chemotherapeutic treatment, radiation treatment) or a diagnostic procedure.

As used herein, the term “hair discoloration” generally refers to a changing in hair color. Examples of hair discoloration include, but are not limited to, a dulling of a subject's hair color, a graying of a subject's hair color (e.g., from blonde color to gray color, from brown color to gray color, from black color to gray color), a whitening of a subject's hair color (e.g., from blonde color to white color, from brown color to white color, from black color to white color).

As used herein, the term “mTOR pathway,” “mTOR signaling pathway,” or “mTOR associated pathway” refers generally to biological (e.g., molecular, genetic, cellular, biochemical, pharmaceutical, environmental) events (e.g., cellular pathways, cellular mechanisms, cellular cascades) involving the mTOR gene and/or the mTOR protein. Examples of components of the mTOR pathway include, but are not limited to, TSC-1, TSC-2, TSC-1/TSC-2 complex, raptor, rictor, GβL, FKBP12, Rheb, mTOR, S6K, and 4EBP-1.

As used herein, the term “mTOR function” refers generally to any type of cellular event for which mTOR is involved (e.g., DNA based activity, mRNA based activity, protein based activity; phosphorylation; associated pathway activity) (e.g., mTOR activity, FRAP1/mTOR expression, mTOR protein level, mTOR stability).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “effective amount” refers to the amount of a composition (e.g., inhibitor of mTOR) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., compositions of the present invention) to a subject (e.g., a subject or in vivo, in vitro, or ex vivo cells, tissues, and organs). Exemplary routes of administration to the human body can be through the eyes (ophthalmic), mouth (oral), skin (transdermal, topical), nose (nasal), lungs (inhalant), oral mucosa (buccal), ear, by injection (e.g., intravenously, subcutaneously, intratumorally, intraperitoneally, etc.) and the like.

As used herein, the term “co-administration” refers to the administration of at least two agent(s) (e.g., mTOR siRNAs or antibodies and one or more other agents) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. In certain embodiments, the antibodies of the present invention are directed toward a mTOR pathway protein (e.g., anti-mTOR, anti-TSC-1, anti-TSC-2, anti-raptor, anti-rictor, anti-GβL, anti-FKBP12, anti-Rheb, anti-S6K, and anti-4EBP-1).

As used herein, the term “antibody fragments” refers to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments. The antibody fragments preferably retain at least part of the hinge and optionally the CH1 region of an IgG heavy chain. In other preferred embodiments, the antibody fragments comprise at least a portion of the CH2 region or the entire CH2 region. In certain embodiments, the antibody fragments of the present invention are directed toward a mTOR pathway protein.

As used herein, the term “functional fragment”, when used in reference to a monoclonal antibody, is intended to refer to a portion of the monoclonal antibody which still retains a functional activity. A functional activity can be, for example, antigen binding activity or specificity. Monoclonal antibody functional fragments include, for example, individual heavy or light or light chains and fragments thereof, such as VL, VH and Fd; monovalent fragments, such as Fv, Fab, and Fab′; bivalent fragments such as F(ab′)₂; single chain Fv (scFv); and Fc fragments. Such terms are described in, for example, Harlowe and Lane, Antibodies: A Laboratory Manual, Cold Spring Harbor Laboratory, New York (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, Second Ed., Wiley-Liss, Inc., New York, N.Y. (1990), all of which are herein incorporated by reference. The term functional fragment is intended to include, for example, fragments produced by protease digestion or reduction of a monoclonal antibody and by recombinant DNA methods known to those skilled in the art.

As used herein, “humanized” forms of non-human (e.g., murine) antibodies are chimeric antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are generally made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody may also comprise at least a portion of an immunoglobulin constant region (Fc), typically that of a human immunoglobulin. Examples of methods used to generate humanized antibodies are described in U.S. Pat. No. 5,225,539 to Winter et al. (herein incorporated by reference). In certain embodiments, the present invention employs humanized anti-MEK4 pathway protein antibodies.

As used herein, the term “hypervariable region” refers to the amino acid residues of an antibody which are responsible for antigen-binding. The hypervariable region comprises amino acid residues from a “complementarity determining region” or “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196: 901-917 (1987), hereby incorporated by reference in its entirety). “Framework” or “FR” residues are those variable domain residues other than the hypervariable region residues as defined herein.

As used herein, the term “siRNAs” refers to small interfering RNAs. In some embodiments, siRNAs comprise a duplex, or double-stranded region, which can be in the form of a hairpin of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants. In certain embodiments, the siRNAs target an mTOR pathway nucleic acid, such as the mRNA that encodes one of the mTOR pathway proteins.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., mTOR antibody) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin including the scalp and mucosal cells and tissues (e.g., alveolar, buccal, lingual, masticatory, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives. For examples of carriers, stabilizers and adjuvants,see e.g., Martin, Remington's Pharmaceutical Sciences, 15th Ed., Mack Publ. Co., Easton, Pa. (1975); incorporated herein by reference in its entirety.

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁻ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The follicular life cycle of hair can be divided into 3 phases: anagen, catagen, and telogen. The hair follicle can be divided into 3 regions: the lower segment (bulb and suprabulb), the middle segment (isthmus), and the upper segment (infundibulum). The lower segment extends from the base of the follicle to the insertion of the erector pill muscle. The middle segment is a short section that extends from the insertion of the erector pill muscle to the entrance of the sebaceous gland duct. The upper segment extends from the entrance of the sebaceous gland duct to the follicular orifice.

The histologic features of the hair follicle change continuously and considerably during the hair growth cycle, thereby making follicular anatomy an even more complex entity.

The size of hair follicles varies considerably during the existence of the follicles. Anagen hairs vary in size from large terminal hairs, such as those on the scalp, to the small vellus hairs that cover almost the entire glabrous skin (except palms and soles). Under hormonal influences, the vellus hair follicles in the male beard area usually thicken and darken at puberty. In predisposed individuals, the terminal hairs on the adult scalp can undergo involutional miniaturization (become vellus). Although vellus hairs greatly outnumber terminal hairs, the latter are more apparent visually and have cultural, social, and psychological significance.

Hair graying is a readily visible signs of physiologic aging. While concerns with gray hair may be largely cosmetic, studying the mechanisms of hair graying may yield insights into a variety of questions related to natural aging and melanocyte function. While there are many instances of aberrant pigmentation at birth in mice (see, e.g., Baxter, L. L., et al., Pigment Cell Res 17, 215-24 (2004); Steingrimsson, E., et al., Annu Rev Genet 38, 365-411 (2004); each herein incorporated by reference in their entireties), there are far fewer instances of mice born with normal pigmented hairs that aberrantly gray over time (see, e.g., Schouwey, K., et al., Dev Dyn 236, 282-9 (2007); Nishimura, E. K., et al., Science 307, 720-4 (2005); Chang, S., et al., Nat Genet 36, 877-82 (2004); each herein incorporated by reference in their entireties). As a result, knowledge of the mechanisms of hair graying is limited.

Considerably more work has been done to understand the biology of the compartment in which melanocytes rest: the hair follicle (see, e.g., Blanpain, C., et al., Annu Rev Cell Dev Biol 22, 339-73 (2006); herein incorporated by reference in its entirety). As a result, the progression through the hair cycle, from the resting phase (telogen), to the growing phase (anagen), to the regression phase (catagen) and the molecular mechanisms that underlie this progression are relatively well understood. However, there are gaps in the knowledge, including the identities of signals that cause a hair follicle to enter the cycle or that promote the coordinated cycling of large patches of hair.

In experiments conducted during the course of development of embodiments for the present invention, the effect of mTOR function inhibition was shown, for example, to delay the onset of new hair growth and hair discoloration through increasing the duration of the hair growth cycle. Accordingly, in certain embodiments, the present invention provides methods for treating and/or preventing hair growth cycle-related conditions (e.g., hair graying, hair whitening, undesired change in hair color, hirsutism, hypertrichosis, undesired hair growth, alopecia, undesired hair shedding, iatrogenically-induced hair changes) in a subject, comprising administering to the subject a composition configured to reduce mTOR function (e.g., mTOR activity, FRAP1/mTOR expression, mTOR protein level, mTOR stability) by administering an mTOR inhibiting agent (e.g., rapamyocin (sirolimus), CCI-779 (temsirolimus), everolimus (RAD-001), AP23573, rapamycin analogs (rapalogs), mTOR antibodies, mTOR siRNAs, agents inhibiting mTOR phosphorylation, agents inhibiting interaction of mTOR with its partners, agents inhibiting interaction of mTOR with its substrates) to a subject. Exemplary compositions and methods of the present invention are described in more detail in the following sections: I. mTOR Inhibiting Agents; II. Therapeutics; III. Antibodies; IV. Pharmaceutical Compositions; and V. Kits.

The practice of the present invention employs, unless otherwise indicated, conventional techniques of organic chemistry, pharmacology, molecular biology (including recombinant techniques), cell biology, biochemistry, and immunology, which are within the skill of the art. Such techniques are explained fully in the literature, such as, “Molecular cloning: a laboratory manual” Second Edition (Sambrook et al., 1989); “Oligonucleotide synthesis” (M. J. Gait, ed., 1984); “Animal cell culture” (R. I. Freshney, ed., 1987); the series “Methods in enzymology” (Academic Press, Inc.); “Handbook of experimental immunology” (D. M. Weir & C. C. Blackwell, eds.); “Gene transfer vectors for mammalian cells” (J. M. Miller & M. P. Calos, eds., 1987); “Current protocols in molecular biology” (F. M. Ausubel et al., eds., 1987, and periodic updates); “PCR: the polymerase chain reaction” (Mullis et al., eds., 1994); and “Current protocols in immunology” (J. E. Coligan et al., eds., 1991), each of which is incorporated herein by reference in their entireties.

I. mTOR Inhibiting Agents

mTOR, is a serine/threonine protein kinase that regulates cell growth, cell proliferation, cell motility, cell survival, protein synthesis, and transcription (see, e.g., Hay N, et al. (2004) Genes & Development, 18(16): 1926-45; Beevers C S, et al. (2006) International Journal of Cancer, 119(4):757-64; each herein incorporated by reference in their entireties), mTOR integrates input from multiple upstream pathways, including insulin, growth factors (such as IGF-1 and IGF-2), and mitogens (see, e.g., Hay N, et al. (2004) Genes & Development, 18(16): 1926-45; herein incorporated by reference in its entirety), mTOR also functions as a sensor of cellular nutrient and energy levels and redox status (see, e.g., Hay N, et al. (2004) Genes & Development, 18(16): 1926-45; Tokunaga C, et al. (2004) Biochemical and Biophysical Research Communications, 313:443-46; Sarbassov D D, et al. (2005) Journal of Biological Chemistry, 280(47):39505-509; each herein incorporated by reference in their entireties). The dysregulation of the mTOR pathway is implicated as a contributing factor to various human disease processes (see, e.g., Beevers C S, et al. (2006) International Journal of Cancer, 119(4):757-64; herein incorporated by reference in its entirety), including but not limited to TSC, epilepsy and diabetes. Rapamycin is a bacterial natural product that can inhibit mTOR through association with its intracellular receptor FKBP12 (see, e.g., Huang S, et al. (2001) Drug Resistance Updates, 4:378-91; Huang S, et al. (2003) Cancer Biology and Therapy, 2:222-232; each herein incorporated by reference in its entirety). The FKBP 12-rapamycin complex binds directly to the FKBP 12-Rapamycin Binding (FRB) domain of mTOR (see, e.g., Huang S, et al. (2003) Cancer Biology and Therapy, 2:222-232; incorporated herein by reference in its entirety).

mTOR has been shown to function as the catalytic subunit of two distinct molecular complexes in cells (see, e.g., Wullschleger S, et al. (2006) Cell, 124(3):471-84; incorporated herein by reference in its entirety), mTOR Complex 1 (mTORC1) is composed of mTOR, regulatory associated protein of mTOR (Raptor), and mammalian LST8/G-protein β3-subunit like protein (mLST8/GβL) (see, e.g., Kim D H, et al. (2002) Cell, 110:163-75; Kim D H, et al. (2003) Molecular Cell, 11:895-904; each incorporated herein by reference in their entireties). This complex possesses the classic features of mTOR by functioning as a nutrient/energy/redox sensor and controlling protein synthesis (see, e.g., Kim D H, et al. (2002) Cell, 110: 163-75; Hay N, et al. (2004) Genes & Development, 18(16): 1926-45; each incorporated herein by reference in their entireties). The activity of this complex is stimulated by insulin, growth factors, serum, phosphatidic acid, amino acids (particularly leucine), and oxidative stress (see, e.g., Kim D H, et al. (2002) Cell, 110: 163-75; Sarbassov D D, et al. (2005) Journal of Biological Chemistry, 280(47):39505-509; Fang Y, et al. (2001) Science, 294:1942-45; each incorporated herein by reference in their entireties), mTORC 1 is inhibited by low nutrient/amino acid levels, serum-starvation/growth factor deprivation, reductive stress, and caffeine, rapamycin, farnesylthiosalicylic acid (FTS) and curcumin (see, e.g., Kim D H, et al. (2002) Cell, 110: 163-75; Sarbassov D D, et al. (2005) Journal of Biological Chemistry, 280(47):39505-509; McMahon LP, et al. (2005) Molecular Endocrinology, 19(1): 175-83; Beevers C S, et al. (2006) International Journal of Cancer, 119(4):757-64: each incorporated herein by reference in their entireties). Two characterized targets of mTORC 1 are p70-S6 Kinase 1 (S6K1) and eukaryotic initiation factor 4E (eIF4E) binding protein 1 (4E-BP1) (see, e.g., Hay N, et al. (2004) Genes & Development, 18 (16): 1926-45; incorporated herein by reference in its entirety), mTORC 1 phosphorylates S6K1 on at least two residues, with the most critical modification occurring on threonine389 (see, e.g., Saitoh M, et al. (2002) Journal of Biological Chemistry, 277:20104-112; Pullen N, et al. (1997)FEBS Letters, 410:78-82; incorporated herein by reference in its entirety). This event stimulates the subsequent phosphorylation of S6K1 by PDK1 (see, e.g., Pullen N, et al. (1997) FEBS Letters, 410:78-82; Pullen N, et al. (1998) Science, 279:707-10; each incorporated herein by reference in their entireties). Active S6K1 can in turn stimulate the initiation of protein synthesis through activation of S6 Ribosomal protein (a component of the ribosome) and other components of the translational machinery (see, e.g., Peterson R, et al. (1998) Current Biology, 8:R248-50; incorporated herein by reference in its entirety). S6K1 can also participate in a positive feedback loop with mTORC 1 by phosphorylating mTOR's negative regulatory domain at threonine2446 and serine2448, events which appear to be stimulatory in regards to mTOR activity (see, e.g., Chiang G G, et al. (2005) Journal of Biological Chemistry, 280:25485-90; Holz M K, et al. (2005) Journal of Biological Chemistry, 280:26089-93; each incorporated herein by reference in their entireties), mTORC 1 has been shown to phosphorylate at least four residues of 4E-BP1 in a hierarchial manner (see, e.g., Gingras A C, et al. (1999) Genes & Development, 13:1422-37; Huang S, et al. (2001) Drug Resistance Updates, 4:378-91; Mothe-Satney I, et al. (2000) Journal of Biological Chemistry, 275:33836-43; each incorporated herein by reference in their entireties). Non-phosphorylated 4E-BP1 binds tightly to the translation initiation factor eIF4E, preventing it from binding to 5′-capped mRNAs and recruiting them to the ribosomal initiation complex (see, e.g., Hay N, et al. (2004) Genes & Development, 18(16): 1926-45; Pause A, et al. (1994) Nature, 371:762-67; each incorporated herein by reference in their entireties). Upon phosphorylation by mTORC 1, 4E-BP1 releases eIF4E, allowing it to perform its function (see, e.g., Pause A, et al. (1994) Nature, 371:762-67; incorporated herein by reference in its entirety). The activity of mTORC 1 appears to be regulated through a dynamic interaction between mTOR and Raptor, one which is mediated by GβL (see, e.g., Kim D H, et al. (2002) Cell, 110: 163-75; Kim D H, et al. (2003) Molecular Cell, 11:895-904; each incorporated herein by reference in their entireties). Raptor and mTOR share a strong N-terminal interaction and a weaker C-terminal interaction near mTOR's kinase domain (see, e.g., Kim D H, et al. (2002) Cell, 110:163-75; incorporated herein by reference in its entirety). When stimulatory signals are sensed, such as high nutrient/energy levels, the mTOR-Raptor C-terminal interaction is weakened, allowing mTOR kinase activity to be turned on (see, e.g., Kim D H, et al. (2002) Cell, 110: 163-75; incorporated herein by reference in its entirety). When stimulatory signals are withdrawn, such as low nutrient/energy levels, the mTOR-Raptor C-terminal interaction is strengthened, essentially shutting off mTOR kinase function (see, e.g., Kim D H, et al. (2002) Cell, 110:163-75; incorporated herein by reference in its entirety).

mTOR Complex 2 (mTORC2) is composed of mTOR, rapamycin-insensitive companion of mTOR (Rictor), GβL, and mammalian stress-activated protein kinase interacting protein 1 (mSIN1)(see, e.g., Frias M A,, et al. (2006) Current Biology, 16(18):1865-70; Sarbassov D D, et al. (2004) Current Biology, 14:1296-1302; each incorporated herein by reference in their entireties), mTORC2 has been shown to function as an important regulator of the cytoskeleton through its stimulation of F-actin stress fibers, paxillin, RhoA, Racl, Cdc42, and protein kinase C α (PKCα)(see, e.g., Sarbassov D D, et al. (2004) Current Biology, 14:1296-1302; incorporated herein by reference in its entirety). However, an unexpected function of mTORC2 is its role as “PDK2.” mTORC2 phosphorylates the serine/threonine protein kinase Akt/PKB at serine473, an event which stimulates Akt phosphorylation at threonine308 by PDK1 and leads to full Akt activation (see, e.g., Sarbassov D D, et al. (2004) Current Biology, 14:1296-1302; Stephens L, et al. (1998) Science, 279:710; each incorporated herein by reference in their entireties), mTORC2 appears to be regulated by insulin, growth factors, serum, and nutrient levels (see, e.g., Frias M A, et al. (2006) Current Biology, 16(18): 1865-70; incorporated herein by reference in its entirety). Originally, mTORC2 was identified as a rapamycin-insensitive entity, as acute exposure to rapamycin did not affect mTORC2 activity or Akt phosphorylation (see, e.g., Sarbassov D D, et al. (2004) Current Biology, 14:1296-1302; Sarbassov D D, et al. (2005) Science, 307:1098-1101; each incorporated herein by reference in their entireties). However, subsequent studies have shown that chronic exposure to rapamycin, while not effecting pre-existing mTORC2s, can bind to free mTOR molecules, thus inhibiting the formation of new Complex 2s (see, e.g., Sarbassov D D, et al. (2006) Molecular Cell, 22(2): 159-68; incorporated herein by reference in its entirety). It has also been shown that curcumin can inhibit the mTORC2-mediated phosphorylation of Akt/PKB at serine473, with subsequent loss of PDK1- mediated phosphorylation at threonine308 (see, e.g., Beevers C S, et al. (2006) International Journal of Cancer, 119(4):757-64; incorporated herein by reference in its entirety).

The present invention provides agents capable of inhibiting mTOR function (e.g., mTOR activity, FRAP1/mTOR expression, mTOR protein level, mTOR stability). The present invention is not limited to a particular type of agent capable of inhibiting mTOR expression. In some embodiments, the mTOR inhibiting agent is an agent that inhibits any part of the pathways associated with mTOR function (e.g., mTOR activity, FRAP1/mTOR expression, mTOR protein level, mTOR stability) (e.g., IRS1, PI3K, PDK1, Akt, PKC, Rac, Rho, LKB1, Rheb, FKBP, mTOR, mLST8/GβL, S6K, S6, 4EBP1, rS6, eIF4E, eIF3, eIF4A, eIF4G, eIF4B, raptor, Vps34, rictor, PTEN, GSK3, LKB1, AMPK, RTP801/L HIF1, REDD1, TSC1, TSC2). In some embodiments, the mTOR inhibiting agent is rapamycin and rapamycin derivatives. In some embodiments, the mTOR inhibiting agent is rapamyocin (sirolimus), CCI-779 (temsirolimus), everolimus (RAD-001), AP23573, rapamycin analogs (rapalogs), mTOR antibodies, mTOR siRNAs, agents inhibiting mTOR phosphorylation, agents inhibiting interaction of mTOR with its partners, or agents inhibiting interaction of mTOR with its substrates.

Rapamycin (sirolimus [Rapamune]) is a commercially available immunosuppressant that forms an inhibitory complex with the immunophilin FKBP12, which then binds to and inhibits the ability of mTOR to phosphorylate downstream substrates, such as the S6Ks and 4EBPs. It is marketed as an immunosuppressant because of its propensity to inhibit T-cell proliferation, and has been approved for use in this therapeutic setting in the United States since 2001. Two derivatives of rapamycin, RAD001 (everolimus [Certican]) and a prodrug for rapamycin, CCI-779 or temsirolimus, are in clinical development in a number of therapeutic indications, including oncology (see, e.g., Chapman T, et al., Drugs 2004: 64: 861-872; Temsirolimus: CCI 779, CCI-779, cell cycle inhibitor-779. Drugs R D 2004; 5: 363-367; each herein incorporated by reference in their entireties). Animal studies have demonstrated the ability of rapamycin to inhibit the aberrant growth of TSC-deficient cells in vitro and to induce apoptosis of renal tumors in animal models of TSC (see, e.g., Kenerson H, et al., Pediatr Res 2005; 57: 67-75; herein incorporated by reference in its entirety).

In some embodiments, the present invention provides compositions for treating, preventing and researching (e.g., identification of new drugs) hair growth cycling-related conditions (e.g., hair graying, hair whitening, undesired change in hair color, hirsutism, hypertrichosis, undesired hair growth, alopecia, undesired hair shedding, iatrogenically-induced hair changes), wherein the compositions comprise mTOR inhibiting agents (e.g., rapamyocin (sirolimus), CCI-779 (temsirolimus), everolimus (RAD-001), AP23573, rapamycin analogs (rapalogs), mTOR antibodies, mTOR siRNAs, agents inhibiting mTOR phosphorylation, agents inhibiting interaction of mTOR with its partners, agents inhibiting interaction of mTOR with its substrates). In some embodiments, such compositions comprising mTOR inhibiting agents (e.g., rapamyocin (sirolimus), CCI-779 (temsirolimus), everolimus (RAD-001), AP23573, rapamycin analogs (rapalogs), mTOR antibodies, mTOR siRNAs, agents inhibiting mTOR phosphorylation, agents inhibiting interaction of mTOR with its partners, agents inhibiting interaction of mTOR with its substrates) are used to, for example, prevent hair follicle cycling, and/or promote the retention of older black hairs over the generation of new potentially gray hairs. In some embodiments, such compositions comprising mTOR inhibiting agents (e.g., rapamyocin (sirolimus), CCI-779 (temsirolimus), everolimus (RAD-001), AP23573, rapamycin analogs (rapalogs), mTOR antibodies, mTOR siRNAs, agents inhibiting mTOR phosphorylation, agents inhibiting interaction of mTOR with its partners, agents inhibiting interaction of mTOR with its substrates) are used to treat undesirable hair growth (e.g., hirsutism, hypertrichosis, iatrogenically-caused hair growth, hair growth considered undesirable for cosmetic reasons). In some embodiments, such compositions comprising mTOR inhibiting agents (e.g., rapamyocin (sirolimus), CCI-779 (temsirolimus), everolimus (RAD-001), AP23573, rapamycin analogs (rapalogs), mTOR antibodies, mTOR siRNAs, agents inhibiting mTOR phosphorylation, agents inhibiting interaction of mTOR with its partners, agents inhibiting interaction of mTOR with its substrates) are used to slow or prevent hair loss (e.g., alopecia, iatrogenically-caused hair loss, hair loss considered undesirable for cosmetic reasons).

### II. Therapeutics

In preferred embodiments, the present invention provides a method of preventing, treating and/or researching hair growth cycle-related conditions (e.g., hair graying, hair whitening, undesired change in hair color, hirsutism, hypertrichosis, undesired hair growth, alopecia, undesired hair shedding, iatrogenically-induced hair changes) in subjects comprising altering (e.g., reducing, inhibiting) mTOR function (e.g., mTOR activity, FRAP1/mTOR expression, mTOR protein level, mTOR stability). In some embodiments, altering mTOR function comprises providing to a cell (e.g., hair follicle cell, bulge cell, hair follicle stem cell) a composition comprising a mTOR inhibiting agent (e.g., rapamyocin (sirolimus), CCI-779 (temsirolimus), everolimus (RAD-001), AP23573, rapamycin analogs (rapalogs), mTOR antibodies, mTOR siRNAs, agents inhibiting mTOR phosphorylation, agents inhibiting interaction of mTOR with its partners, agents inhibiting interaction of mTOR with its substrates) or candidate mTOR inhibiting agent. In some embodiments, altering mTOR function comprises altering (e.g., reducing, inhibiting) components (e.g., associated pathway agents) that interact with, influence, or are affected by mTOR (e.g., IRS1, PI3K, PDK1, Akt, PKC, Rac, Rho, LKB1, Rheb, FKBP, mTOR, mLST8/GβL, S6K, S6, 4EBP1, rS6, eIF4E, eIF3, eIF4A, eIF4G, eIF4B, raptor, Vps34, rictor, PTEN GSK3, LKB1, AMPK, RTP80/L, HIF1, REDD1, TSC1, TSC2). In some embodiments, altering mTOR function comprises altering (e.g., reducing, inhibiting) genes or proteins normally upregulated in response to elevated mTOR function. In some embodiments, altering mTOR function comprises altering (e.g., increasing the expression of, increasing the activity of, increasing the protein level of) genes or proteins normally downregulated in response to elevated mTOR function. In some embodiments, altering mTOR function involves a combination of several approaches, including but not limited to, altering mTOR function (e.g., mTOR activity, FRAP1/mTOR expression, mTOR protein level, mTOR stability), altering mTOR associated pathways, and altering transcription, translation, or stability of mTOR-responsive genes or proteins.

The present invention is not limited by the type of inhibitor used to inhibit mTOR function (e.g., mTOR activity, FRAP1/mTOR expression, mTOR protein level, mTOR stability) for treating and/or preventing hair cycling-related conditions (e.g., hair graying, hair whitening, undesired change in hair color, hirsutism, hypertrichosis, undesired hair growth, alopecia, undesired hair shedding, iatrogenically-induced hair changes). Indeed, any compound, pharmaceutical, small molecule or agent (e.g., antibody, protein or portion thereof) that can alter mTOR function (e.g., mTOR activity, FRAP1/mTOR expression, mTOR protein level, mTOR stability) is contemplated to be useful in the methods of the present invention. In some embodiments, inhibitors used in altering mTOR function (e.g., mTOR activity, FRAP1/mTOR expression, mTOR protein level, mTOR stability) include, but are not limited to, rapamycin.

In some embodiments, altering mTOR function (e.g., mTOR activity, FRAP1/mTOR expression, mTOR protein level, mTOR stability) comprises providing to a cell (e.g., hair follicle cell, bulge cell, hair follicle stem cell) mTOR specific siRNAs. In some embodiments, altering mTOR function comprises providing to a cell siRNAs specific for components of pathways associated with mTOR function. In some embodiments, altering mTOR function comprises providing to a cell siRNAs specific for mTOR and/or agents associated with mTOR associated pathways (e.g., IRS 1, PI3K, PDK1, Akt, PKC, Rac, Rho, LKB1, Rheb, FKBP, mTOR, mLST8/GβL, S6K, S6, 4EBP1, rS6, eIF4E, eIF3, eIF4A, eIF4G, eIF4B, raptor, Vps34, rictor, PTEN, GSK3, LKB1, AMPK, RTP801/L, HIF1, REDD1, TSC1, TSC2). The present invention is not limited by the siRNA used. For example, in some embodiments, the present invention provides siRNAs of about 18-25 nucleotides long, 19-23 nucleotides long, or even more preferably 20-22 nucleotides long. The siRNAs may contain from about two to four unpaired nucleotides at the 3′ end of each strand. In preferred embodiments, at least one strand of the duplex or double-stranded region of a siRNA is substantially homologous to or substantially complementary to a target RNA molecule (e.g., IRS1, PI3K, PDK1, Akt, PKC, Rac, Rho, LKB1, Rheb, FKBP, mTOR, mLST8/GβL, S6K, S6, 4EBP1, rS6, eIF4E, eIF3, eIF4A, eIF4G, eIF4B, raptor, Vps34, rictor, PTEN, GSK3, LKB1, AMPK, RTP801/L, HIF1, REDD1, TSC1, TSC2). The present invention is not limited by the target RNA molecule/sequence. Indeed, a variety of target sequences are contemplated to be useful in the present invention including, but not limited to, 18-25 nucleotide stretches of the mTOR mRNA sequence (see, e.g., NCBI Accession No. NM_{—}004958 for Homo sapiens mTOR).

In some embodiments, altering mTOR function (e.g., mTOR activity, FRAP1/mTOR expression, mTOR protein level, mTOR stability) comprises providing to the cell (e.g., hair follicle cell, bulge cell, hair follicle stem cell) an antibody specific for mTOR, or an antibody specific for mTOR associated pathways. In some embodiments, the antibody reduces activity of mTOR in the cell. In some embodiments, altering mTOR function in the cell sensitizes the cell to an additional form of therapeutic. In some embodiments, altering mTOR function prevents hair follicle cycling, and/or promotes the retention of older black hairs over the generation of new potentially gray hairs. In some embodiments, altering mTOR function prevents undesirable hair growth (e.g., hirsutism, hypertrichosis, iatrogenically-caused hair growth, hair growth considered undesirable for cosmetic reasons). In some embodiments, altering mTOR function prevents or slows hair loss (e.g., alopecia, iatrogenically-caused hair loss, hair loss considered undesirable for cosmetic reasons). In some embodiments, the present invention also provides a method of treating a subject undergoing a condition associated with hair discoloration comprising providing a composition comprising an inhibitor of mTOR; and administering the composition to the subject under conditions such that mTOR function is altered.

The present invention is not limited to a particular type or kind of condition associated with hair growth cycling (e.g., hair graying, hair whitening, undesired change in hair color, hirsutism, hypertrichosis, undesired hair growth, alopecia, undesired hair shedding, iatrogenically-induced hair changes). Examples include, but are not limited to, aging, the effects of chemotherapy treatment, the effects of radiation treatment, the effects of organ transplant (e.g., bone marrow transplant) treatment, alopecia areata, genetic predisposition, hormonal imbalance, androgen imbalance, exposure to pollutants, exposure to toxins, and chemical exposure. In some embodiments, the composition comprising an inhibitor of mTOR is co-administered with an agent configured to treat hair discoloration (e.g., cosmetic hair dye).

In some embodiments, the composition comprising an inhibitor of mTOR is co-administered with an agent configured to treat hair growth, thickness, texture, lustre, or shedding. In some embodiments, the composition comprising an inhibitor of mTOR is co-administered with an agent affecting hair graying, hair whitening, undesired change in hair color, hirsutism, hypertrichosis, undesired hair growth, alopecia, undesired hair shedding, or iatrogenically-induced hair changes (e.g., hair dyes, hair bleaches, oral contraceptives, Spironolactone (Aldactone), Flutamide (Eulexin), Cyproterone (Diane-35), Cyproterone acetate, corticosteroids, Dexamethasone (Decadron, Dexasone), Prednisone (Deltasone, Sterapred, Orasone), Finasteride, Eflornithine hydrochloride (Vaniqa), Metformin (Glucophage), dehydroepiandrosterone sulfate (DHEA-S), testosterone, danazol, anabolic steroids, phenytoin, minoxidil, diazoxide, cyclosporine, streptomycin, psoralen, penicillamine, high-dose corticosteroids, metyrapone, phenothiazines, acetazolamide, and hexachlorobenzene, Corticosteroids, Dilantin, streptomycin, hexachlorobenzene, penicillamine, cyclosporine, diazoxide, minoxidil, heavy metals, sodium tetradecyl sulfate, acetazolamide, interferon, Squaric acid dibutylester and diphencyprone, Cyclosporine (Sandimmune, Neoral), Methoxsalen (8-MOP, Oxsoralen), Anthralin, Clobetasol propionate (Temovate), Prednisone (Deltasone, Meticorten, Sterapred), Triamcinolone acetonide suspension (Kenalog), Betamethasone dipropionate cream 0.05% (Diprosone), Minoxidil (Rogaine), Finasteride (Propecia)).

In some embodiments, the composition comprising an inhibitor of mTOR is co-administered with an anti-cancer agent (e.g., chemotherapeutic) (e.g., for purposes of preventing and/or treating undesired hair loss resulting from treatment with an anti-cancer agent). The present invention is not limited by type of anti-cancer agent co-administered. Indeed, a variety of anti-cancer agents are contemplated to be useful in the present invention including, but not limited to, Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourca (BCNU); N-(2-chloroethyl)-N′-cyclohex-yl-N-nitrosourca (CCNU); N-(2-chloroethyl)-N′-(trans-4-methylcyclohexyl-N-nitrosourea (MeCCNU); N-(2-chloroethyl)-N′-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; Iosoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda).

Other anti-cancer agents include: Antiproliferative agents (e.g., Piritrexim Isothionate), Antiprostatic hypertrophy agent (e.g., Sitogluside), Benign prostatic hypertrophy therapy agents (e.g., Tamsulosin Hydrochloride), Prostate growth inhibitor agents (e.g., Pentomone), and Radioactive agents: Fibrinogen I 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; Triolein I 131.

Another category of anti-cancer agents is anti-cancer Supplementary Potentiating Agents, including: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL.

Still other anticancer agents are those selected from the group consisting of: annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-C1-2′deoxyadenosine; Fludarabine-PO₄; mitoxantrone; mitozolomide; Pentostatin; and Tomudex.

One particularly preferred class of anticancer agents are taxanes (e.g., paclitaxel and docetaxel). Another important category of anticancer agent is annonaceous acetogenin. Other cancer therapies include hormonal manipulation. In some embodiments, the anti-cancer agent is tamoxifen or the aromatase inhibitor arimidex (i.e., anastrozole).

### III. Antibodies

The present invention provides isolated antibodies. In preferred embodiments, the present invention provides monoclonal antibodies that specifically bind to the mTOR biomarkers (e.g., IRS1, PI3K, PDK1, Akt, PKC, Rac, Rho, LKB1, Rheb, FKBP, mTOR, mLST8/GβL, S6K, S6, 4EBP1, rS6, eIF4E, eIF3, eIF4A, eIF4G, eIF4B, raptor, Vps34, rictor, PTEN, GSK3, LKB1, AMPK, RTP801/L, HIF1, REDD1, TSC1, TSC2). Examples include, but are not limited to, monoclonal antibody against mTOR (e.g., Abcam#s: ab2732, ab2833, ab19207, ab1093, ab34758, ab25880, ab32028), monoclonal antibody against PI3K (e.g., Abcam#s: ab249, ab250, ab40776, ab32089, ab32401), monoclonal and polyclonal antibodies against Akt (e.g., Abcam#s: ab18785, ab28821, ab27773, ab38449, ab39421, ab28422, ab31391, ab35738, ab24831, ab24818), monoclonal antibody against LKB1 (e.g., Abcam#s: ab15095, ab37219), monoclonal and polyclonal antibodies against AMPK (e.g., Abcam#s: ab31958, ab32508, ab32382, ab32112, ab32047, ab3759, ab39644, ab23875, ab3900, ab3760), monoclonal and polyclonal antibodies against TSC1, TSC2, and TSC1/TSC2 (e.g., Abcam#s: ab32936, ab25881, ab25882, ab40872, ab25883), polyclonal antibodies against Rheb (e.g., Abcam#s: ab25873, ab25976), monoclonal and polyclonal antibodies against S6K1 (e.g., Abcam#s: ab19327, ab19279, ab28554, ab24490, ab19380, ab2571, ab24488, ab32529, ab9367, ab36864, ab32525, ab32359, ab9366, ab5231), monoclonal antibody against rS6 (e.g., Abcam# ab 10128), monoclonal and polyclonal antibodies against 4EBP 1 (e.g., ab37225, ab32130, ab32024, ab25872, ab2606, ab27792), and antibodies against eIF4E. These antibodies, and others, find use in the diagnostic and therapeutic methods described herein.

### IV. Pharmaceutical Compositions

The present invention further provides pharmaceutical compositions (e.g., comprising an inhibitor of mTOR function described herein). The pharmaceutical compositions of the present invention may be administered in a number of ways depending upon whether local or systemic treatment is desired and upon the area to be treated. Administration may be topical (including through direct contact with hair follicles), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids, semi-solids, monophasic compositions, multiphasic compositions (e.g., oil-in-water, water-in-oil), foams microspohnges, liposomes, nanoemulsions, aerosol foams, polymers, fullerenes, and powders (see, e.g., Taglietti et al (2008) Skin Ther. Lett. 13:6-8). Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carder compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing may be dependent on severity and responsiveness of the condition or disease state (e.g., stage of the hair discoloration, state of hair growth cycle, level of hair growth, grading of hair growth using modified Ferriman-Gallwey scoring system) to be treated, with the course of treatment lasting from several days to several months, or until a cure is effected or a diminution of the condition or disease state is achieved. Optimal dosing schedules can be calculated from measurements of drug accumulation in the body of the patient. The administering professional (e.g., physician) can easily determine optimum dosages, dosing methodologies and repetition rates. Optimum dosages may vary depending on the relative potency of the agent (e.g., molecule, oligonucleotide, siRNA, antibody), and can generally be estimated based on EC₅₀ₛ found to be effective in in vitro and in vivo animal models or based on the examples described herein. In general, dosage is from 0.01 μg to 100 g per kg of body weight, and may be given once or more daily, weekly, monthly or yearly. The administering professional can estimate repetition rates for dosing based on measured residence times and concentrations of the drug in bodily fluids or tissues. Following successful treatment, it may be desirable to have the subject undergo maintenance therapy to prevent the recurrence of the disease state, wherein the treatment (e.g., molecule, siRNA or antibody) is administered in maintenance doses, ranging from 0.01 μg to 100 g per kg of body weight, once or more daily, to once every 20 years.

In experiments conducted during the course of the development of the embodiments of the present invention, rapamycin was shown to prevent hair graying in subjects predisposed to hair graying. Rapamycin was further shown to prevent hair growth following depilation. The present invention is not limited to a particular amount of rapamycin for administration to a subject (e.g., 100 mg/kg/day, 90 mg/kg/day, 80 mg/kg/day, 50 mg/kg/day, 25 mg/kg/day, 15 mg/kg/day, 10 mg/kg/day, 5 mg/kg/day, 1 mg/kg/day, 0.1 mg/kg/day, 0.01 mg/kg/day). In some embodiments, the amount of rapamycin for administration is between 1 and 30 mg/day (e.g., 5-15 mg/kg/day) (e.g. 7 mg/kg/day). In some embodiments, the rapamycin is continuously administered to a subject.

### V. Kits

In yet other embodiments, the present invention provides kits for the prevention and/or treatment of hair growth cycling-related conditions (e.g., hair graying, hair whitening, undesired change in hair color, hirsutism, hypertrichosis, undesired hair growth, alopecia, undesired hair shedding, iatrogenically-induced hair changes). In some embodiments, the kits contain antibodies specific for mTOR pathway members or biomarkers (e.g., IRS1, PI3K, PDK1, Akt, PKC, Rac, Rho, LKB 1, Rheb, FKBP, mTOR, mLST8/GβL, S6K, S6, 4EBP1, rS6, eIF4E, eIF3, eIF4A, eIF4G, eIF4B, raptor, Vps34, rictor, PTEN, GSK3, LKB1, AMPK, RTP801/L, HIF1, REDD1, TSC1, TSC2). In some embodiments, the kits contain mTOR inhibiting agents (e.g., rapamyocin (sirolimus), CCI-779 (temsirolimus), everolimus (RAD-001), AP23573, rapamycin analogs (rapalogs), mTOR antibodies, mTOR siRNAs, agents inhibiting mTOR phosphorylation, agents inhibiting interaction of mTOR with its partners, agents inhibiting interaction of mTOR with its substrates). In some embodiments, the kits further contain detection reagents and buffers. In other embodiments, the kits contain reagents specific for the detection of nucleic acids (e.g., DNA, RNA, mRNA or cDNA, oligonucleotide probes or primers, labeled nucleic acids, reagents for detecting or visualizing labeled nucleic acids). In other embodiments, the kits contain reagents specific for the detection of proteins (e.g., antibodies, conjugated antibodies, labeled antibodies, reagents to detect or visualize antibodies). In preferred embodiments, the kits contain all of the components necessary and/or sufficient to perform a detection assay, including all controls, directions for performing assays, and any necessary software for analysis and presentation of results.

### Experimental

The following examples are provided in order to demonstrate and further illustrate certain some embodiments and aspects of the present invention and are not to be construed as limiting the scope thereof.

### EXAMPLE I

This example describes the materials and methods used for Example. II.

Mice: DCT-LacZ mice were backcrossed for 3 generations on a C57BL background.

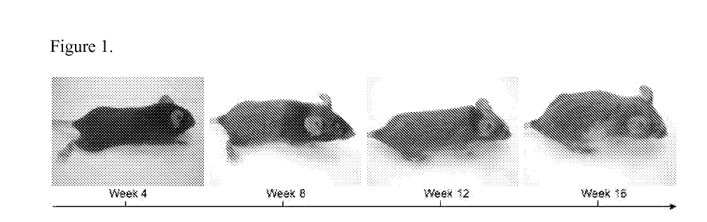

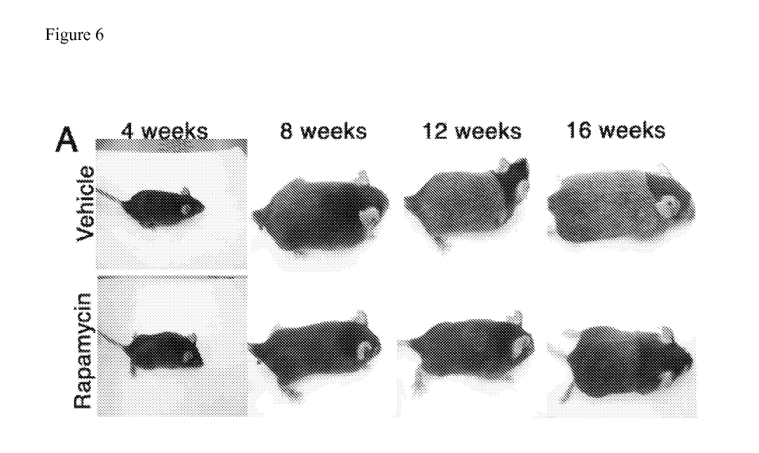

Irradiation and Bone Marrow Transplant: Adult recipient mice were irradiated using a gamma cell irradiator. Mice received two doses of 560 rads each delivered 3 hours apart (FIGS. 1-4, FIG. 7, Table 1). Mice received 1,000,000 whole bone marrow cells isolated from a C57BL donor (FIGS. 1-4, FIG. 7, Table 1). Bone marrow cells were suspended in HBSS free with 2% Bovine Serum and injected via the retro orbital venous sinus. Where indicated, mice were sublethally irradiated with 650 rads and not subjected to bone marrow transplant (FIG. 6).

Rapamycin: Rapamycin (LC Laboratories) was administered by intraperitoneal injection. Rapamycin was reconstituted in ethanol (10 mg/ml or 1 mg/ml) and diluted into a solution of 5% Tween-80 (Sigma) and 5% PEG-400 (Hampton Research). The final volume injected was 200 μg. Dosing and administration regimes were as indicated in Example II.

DCT-LacZ Staining A 2×2 cm patch of skin was harvested with dissecting scissors from euthanized DCT-LacZ mice. The skin patch was washed in phosphate buffer (0.5M Na₂HPO₄, 0.5M NaH₂PO₄, 2 mM MgC₁₂, 0.02% NP-40, 0.01% sodium deoxycolate), and then fixed on ice in 0.2% glutaraldehyde in phosphate buffer for 30 minutes. While being fixed, the skin samples were microwaved in fixation solution on ice for 20 seconds. After fixation, the samples were washed and the adipose layer was removed by dissection. After another washing, the skin samples were stained in 1 mg/ml X-gal in phosphate buffer for 2.5 hours. After staining, samples were washed and roughly 1 mm wide sections were made manually using a razor blade. Sections were mounted in 70% glycerol.

Depilation: Mice were anesthetized by intraperitoneal injection of 80 mg/kg of ketamine and 4 mg/kg of xylene dissolved in HBSS. Final injection volume was 200 μl. The dorsal hair of the mice was then shaved with conventional clippers, and a commercial depilatory cream (Veet®) was applied liberally to an area of approximately 1.5×1.5 cm. After 5 minutes, the cream was washed off the mouse using warm water, and the mouse was gently dried using paper towels.

### EXAMPLE II

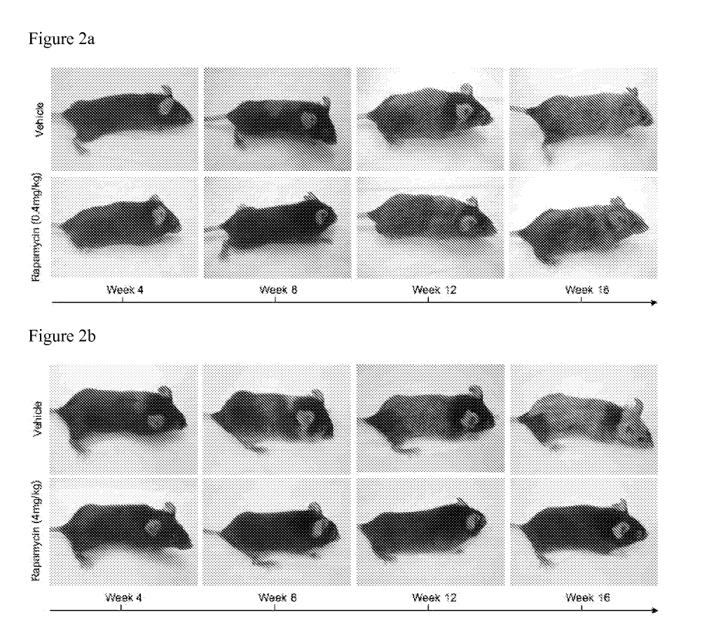

It has been observed that when mice receive a bone marrow transplant, their hair loses pigmentation over time following transplant resulting in mice that have a gray haired appearance (see FIG. 1). To systematically test the ability of rapamycin to alter hair graying, 1 million whole bone marrow cells were transplanted from a C57BL mouse into recipient mice that received intraperitoneal injections of either rapamycin or vehicle control. Due to perceived sensitivity to high doses of rapamycin immediately following transplant, the mice were treated every other day with rapamycin at a dose of 0.4 mg/kg or vehicle control for the first two weeks. After this time period, the mice were treated every day with doses of either 0.4 mg/kg or 4 mg/kg of rapamycin or vehicle control. The onset of graying was delayed and the extent of graying lessened in mice treated with rapamycin at 0.4 mg/kg/day for four months (see FIG. 2a). Hair graying was prevented in mice treated with rapamycin at 4 mg/kg/day for four months (see FIG. 2b). Both the 4 mg/kg/day and 0.4 mg/kg/day mice received the same dose and treatment schedule for the first two weeks after transplant (0.4 mg/kg every two days), indicating that the higher dose of rapamycin's ability to completely prevent hair graying was due to effects that occurred after the first two weeks of treatment.

Experiments were next conducted to investigate how rapamycin delayed hair graying. The Ink4a and ARF tumor suppressor genes encode proteins that promote senescence and that reduce the function of stem cells and other progenitors from a variety of aging mammalian tissues (see, e.g., Kim, W. Y. & Sharpless, N. E., Cell 127, 265-75 (2006); herein incorporated by reference in its entirety). To test whether the graying observed after irradiation was caused by Ink4a/ARF function, 1 million whole bone marrow cells from an Ink4a-ARF heterozygous mouse were irradiated and transplanted into Ink4a-ARF heterozygous or homozygous null recipients. Both the Ink4a-ARF heterozygous and homozygous null recipients grayed similarly, indicating that hair graying in this context was independent of Ink4a and ARF. Additionally, it was observed that rapamycin (0.4 mg/kg) was able to protect the Ink4a-ARF-deficient and heterozygous mice from hair graying equally, indicating that the ability of rapamycin to prevent hair graying is also independent of Ink4a and ARF. These results indicate that rapamycin is not preventing Ink4a-ARF-mediated senescence of melanocyte progenitors.

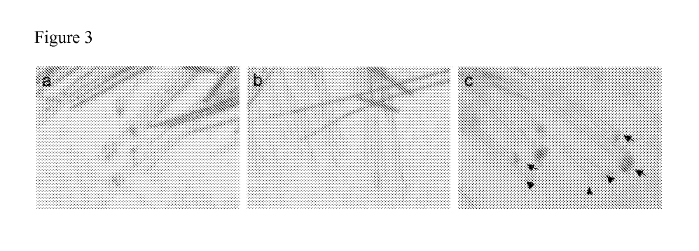

To better understand the effects of rapamycin on melanocyte progenitors, transplantation experiments in mice bearing a DCT-LacZ transgene, which is expressed by melanocyte progenitors in hair follicles, were conducted (see, e.g., Mackenzie, M. A., et al., Dev Biol 192, 99-107 (1997); Nishimura, E. K., et al., Nature 416, 854-60 (2002); each herein incorporated by reference in their entireties). This transgene expresses the bacterial LacZ gene under the control of the melanocyte specific promoter for the gene dopachrome tautomerase. These mice were used as recipients in similar transplants where 1 million whole bone marrow cells were irradiated and transplanted. The mice were treated with 0.4 mg/kg of rapamycin every other day for the first two weeks and then with 4 mg/kg/day after that. These mice were sacrificed at regular intervals, and their skin harvested and stained with X-gal. The skin was sectioned and the number of hair follicles positive for X-gal+ cells quantified, indicating that these follicles still contained melanocyte progenitors. The rapamycin and vehicle treated mice had similar percentages of their hair follicles that contained X-gal+ melanocyte progenitors (See, Table 1 and FIG. 3).

TABLE 1

| | | Week 8 | Week 10 | Week 12 | Week 14 |
|---|---|---|---|---|---|
| Vehicle | Mouse 1 | 84.0 | 51.8 | 38.7 | 36.0 |
| | Mouse 2 | 53.0 | 42.0 | 54.0 | 54.0 |
| | Mouse 3 | | 57.0 | 59.0 | 25.0 |
| | Mouse 4 | | | 46.0 | 14.9 |
| | Mouse 5 | | | | |
| | Mouse 6 | | | | |
| | Average | 68.5 | 50.3 | 49.4 | 32.5 |
| | St. Dev. | 21.9 | 7.6 | 8.9 | 16.7 |
| Rapamycin | Mouse 1 | 77.0 | 19.3 | 54.0 | 53.0 |
| (4 m/kg) | Mouse 2 | 45.0 | 22.0 | 39.0 | 42.0 |
| | Mouse 3 | | 42.0 | 52.0 | 17.0 |
| | Mouse 4 | | 53.0 | | 50.0 |
| | Mouse 5 | | | | |
| | Mouse 6 | | | | |
| | Average | 61.0 | 34.1 | 48.3 | 40.5 |
| | St. Dev. | 22.6 | 16.2 | 8.1 | 16.3 |
| | T-test | 0.8 | 0.2 | 0.9 | 0.5 |

As shown in Table 1, mice were irradiated with 11.2 grays of irradiation and treated with 4 mg/kg/day of rapamycin or vehicle. Animals were sacrificed at each time point and stained with X-gal. The percentage of hair follicles containing DCT-LacZ+ cells was quantified.

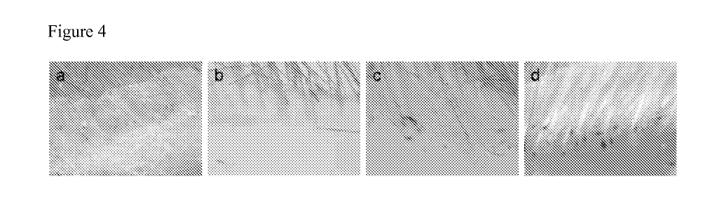

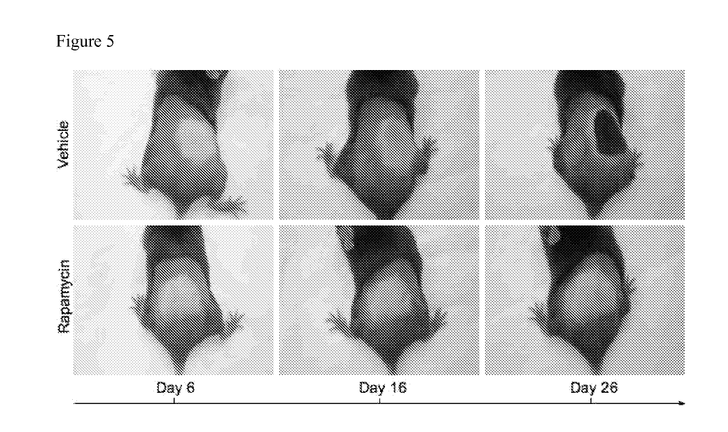

In analyzing the frequency of hair follicles that contained X-gal+ melanocytes, it became apparent that the hair follicles of rapamycin treated animals were less frequently in mature stages of the hair cycle (see FIG. 4). Whereas 56% ( 10/18) of the vehicle treated animals appeared to have hairs in the anagen phase of the hair cycle, only 11% ( 2/18) of rapamycin treated animals had hairs that were in anagen (P=0.0001, Pearson's chi-squared test), suggesting that rapamycin may be slowing or arresting the hair cycle. As such, a region of the lower back of wild-type nonirradiated mice was shaved and depilated. Depilation induced a new hair growth cycle, thereby permitting to test if in this context rapamycin could prevent the onset of or lengthen the duration of the hair cycle. Mice were depilated and starting the day after depilation were treated with either rapamycin (4 mg/kg) or vehicle control (see FIG. 5). Rapamycin delayed the change in skin color that corresponds with production of melanin and progression through anagen by on average 8 days (rapamycin=25.2 days until pigmentation was visible in skin, n=5 versus vehicle=16.7 days until pigmentation was visible in skin n=3, p=0.04). This indicated that rapamycin delayed the entry into a new hair cycle after depilation.

To determine whether rapamycin treatment would affect the emergence of gray hairs after sublethal irradiation in absence of bone marrow cell transplantation, mice were sublethally irradiated with 650 rads and were treated with vehicle (n=5) or with 4 mg/kg of rapamycin (n=5). Representative pictures from each group are shown at 4, 8, 12, and 16 weeks post-irradiation (see FIG. 6). FIG. 6 shows that treatment with rapamycin decreased gray hair in absence of transplantation.

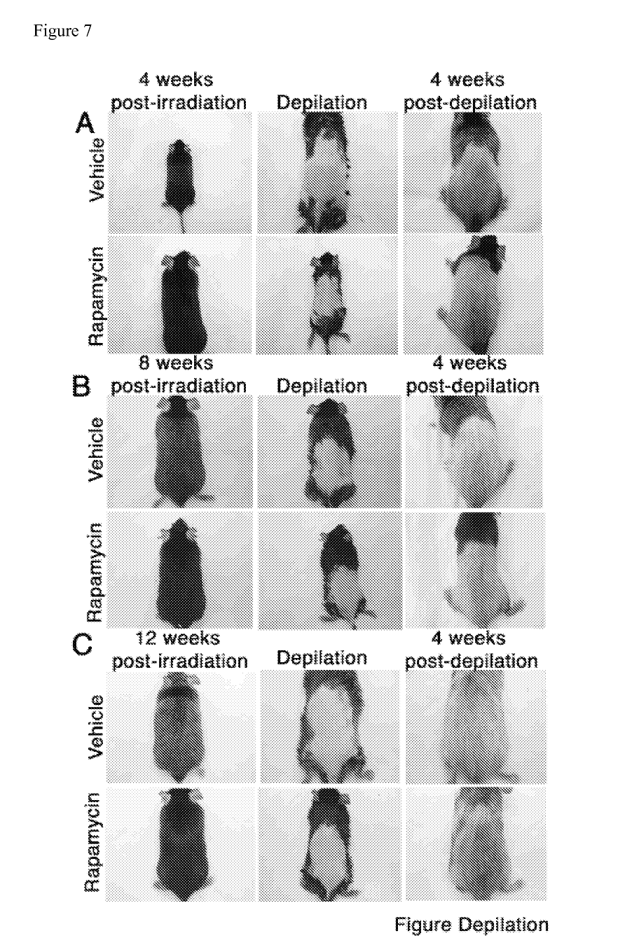

To determine the effect of rapamycin treatment on hair turnover independently of hair pigmentation after lethal irradiation, mice were irradiated using two doses of 560 rads each delivered 3 hours apart and transplanted with 1,000,000 bone marrow cells from a C57BL mouse. Mice were injected with vehicle or 4 mg/kg rapamycin on alternating days for the first 14 days after transplantation and then daily thereafter. At 4, 8 and 12 weeks post-transplantation, groups of vehicle (n=3 at each time point) and rapamycin-treated mice (n=3 at each time point) were depilated and subsequently not administered treatment. Pictures were taken 4 weeks after depilation (see FIG. 7). Representative pictures from each group are shown. FIG. 7 shows that rapamycin reduces the number of gray hairs after irradiation by, for example, reducing hair turnover and not by rescuing hair pigmentation.

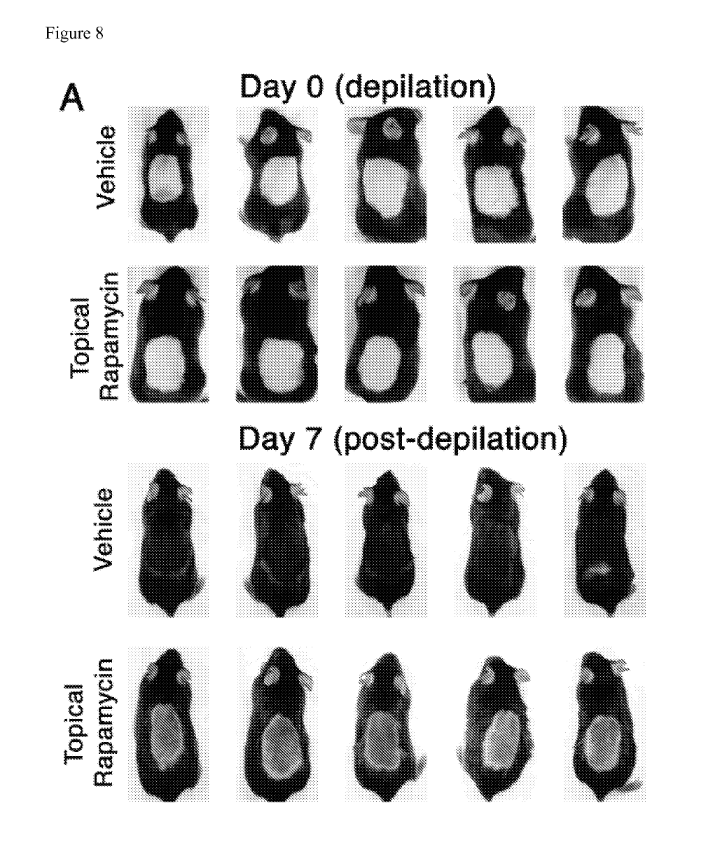

To determine the effect of topical administration of rapamycin on hair growth after depilation, Mice were depilated (day 0) as described herein and a 0.5% topical rapamycin ointment (n=5) or vehicle ointment (n=5) was applied daily for seven days. Topical rapamycin ointment was prepared as previously described (see, e.g., Rauktys et al (2008) BMC Dermatol. 8:1; herein incorporated by reference in its entirety). Briefly, a single dose of a 0.5% ointment was prepared by mixing 20 μl of a 25 mg/ml rapamycin stock solution in absolute ethanol to 100 mg of petroleum jelly. A vehicle ointment was prepared by mixing 20 ul of absolute ethanol to 100 mg of petroleum jelly. Immediately after mixing, the ointment was applied with a gloved finger. As shown in FIG. 8, mice administered topical rapamycin exhibit decreased hair growth.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method of preventing and/or treating a hair growth cycle-related condition, comprising administering to a subject a composition comprising an agent, wherein said agent is designed to increase the duration of a hair's growth cycle, wherein said agent is an mTOR inhibitor.

2. The method of claim 1, wherein said hair growth cycle-related condition is selected from the group consisting of hair discoloration, hirsutism, hypertrichosis, alopecia, undesired hair growth, and undesired hair loss.

3. The method of claim 1, wherein said hair growth cycle-related condition is related to the effects of a medical treatment.

4. The method of claim 4, wherein said medical treatment is selected from the group consisting of chemotherapy treatment, radiation treatment, and organ transplant treatment.

5. The method of claim 2, wherein said hair discoloration is selected from the group consisting of hair graying and hair whitening.

6. The method of claim 1, wherein said agent is selected from the group consisting of rapamycin and a rapamycin derivative.

7. The method of claim 6, wherein said rapamycin derivative is selected from the group consisting of CCI-779, everolimus (RAD-001), and AP23573.

8. The method of claim 2, wherein said hirsutism is related to a condition selected from the group consisting of Polycystic Ovarian Syndrome, Cushing's disease, ovarian tumor, adrenal tumor, congenital adrenal hyperplasia, insulin resistance, excess androgen levels, pregnancy, aging, and obesity.

9. The method of claim 2, further comprising administering at least one additional agent to said subject, wherein said additional agent is selected from the group consisting of an anti-alopecia agent, an anti-hair discoloration agent, an anti-hirsutism agent, an anti-hair growth agent, a hair growth stimulation agent, an anti-hair loss agent, and an anti-hypertrichosis agent.

10. The method of claim 9, wherein said anti-hirsutism agent is selected from the group consisting of an oral contraceptive, spironolactone, flutamide, cyproterone, cyproterone acetate, a corticosteroid, finasteride, eflornithine hydrochloride, and metformin.

11. The method of claim 9, wherein said anti-hair discoloration agent is hair dye.

12. A composition comprising 1) an agent designed to increase the duration of a hair's growth cycle, and 2) at least one additional agent selected from the group consisting of an anti-hair discoloration agent, an anti-hirsutism agent, an anti-alopecia agent, an anti-hair growth agent, a hair stimulation agent, an anti-hair loss agent, and an anti-hypertrichosis agent.

13. The composition of claim 12, wherein said agent designed to increase the duration of a hair's growth cycle is an mTOR inhibitor.

14. The composition of claim 13, wherein said mTOR inhibitor is selected from the group consisting of rapamycin and a rapamycin derivative.

15. The composition of claim 14, wherein said rapamycin derivative is selected from the group consisting of CCI-779, everolimus (RAD-001), and AP23573.

16. The composition of claim 12, wherein said anti-hair discoloration agent is hair dye.

17. The composition of claim 12, wherein said anti-hirsutism agent is selected from the group consisting of an oral contraceptive, spironolactone, flutamide, cyproterone, cyproterone acetate, a corticosteroid, finasteride, eflornithine hydrochloride, and metformin.

18. The composition of claim 12, wherein said composition is configured for topical administration to a subject.
